(19) United States
(12) Patent Application Publication
(10) Pub. No.: US 2026/0263432 A1
(43) Pub. Date: Sep. 10, 2026

(54) COMPOSITIONS FOR SUSTAINED RELEASE OF POORLY SOLUBLE ACTIVE INGREDIENTS

(71) Applicant: Prolevi Bio AB, Lund (SE)

(72) Inventors: Sahil Gupta, Idstein (DE); Daniel Bar-Shalom, Odense C (DK)

(21) Appl. No.: 19/164,089

(22) PCT Filed: Mar. 14, 2024
(86) PCT No.: PCT/EP2024/056768

§ 371 Date: Sep. 10, 2025

(30) Foreign Application Priority Data
Mar. 14, 2023 (EP) 23161909.9
Oct. 24, 2023 (EP) 23205497.3

(51) Int. Cl. A61K 31/4422; A61K 9/20

(52) U.S. Cl. CPC A61K 31/4422; A61K 9/2031; A61K 9/2054; A61K 9/2095

(57) ABSTRACT

The present disclosure relates to the field of sustained release compositions of active ingredients able to enhance the dissolution rate of active ingredients with poor aqueous solubility.

### TECHNICAL FIELD

The present disclosure relates to the field of sustained release compositions of active ingredients able to enhance the dissolution rate of active ingredients with poor aqueous solubility.

### BACKGROUND

Oral administration is one of the most convenient and commonly employed routes of administration for therapeutics. However, one of the major problems with oral administration of drugs is poor bioavailability (BA). This holds especially true for drugs that exhibit low solubility in aqueous medium. Unfortunately, it is the case nowadays that many therapeutics are dosed and administered inefficiently due to poor solubility issues.

Therapeutics having low solubility in aqueous media typically have a rate of absorption that is limited by their rate of dissolution. Hence, strategies to improve the bioavailability of poorly soluble active ingredients often focus on improving the dissolution rate. However, this is often challenging to balance with sustained release compositions that produce a predictable profile of drug release, while enhancing the dissolution rate.

Another reason of ineffective administration of therapeutics is misalignment with day and night oscillation of events in the body. Most bodily functions vary over the course of a 24 hour day, for example, body temperature, sleep-wake cycles, metabolism and blood pressure are examples of functions that are regulated in circadian rhythms. Blood pressure usually decreases during the night and increases in the morning in a cycle that repeats in a 24-hour pattern¹.

Administration of drugs in a manner that their effect matches, or at least approaches and respects the circadian oscillation of events in the body is highly challenging, especially to match bodily events that occur during the night.

Thus, there is currently a need for formulations that allow for improving the dissolution rate of poorly soluble drugs, while providing a sustained release profile that is able to support better therapeutic applications, such as matching circadian rhythms.

### SUMMARY

As indicated above, providing formulations able to improve the dissolution rate of poorly soluble active ingredients while achieving sustained release is highly warranted. The inventors of the present application have discovered sustained-release formulations able to accommodate active ingredients in an amorphous form while being able to provide sustained release over a specific period of time while enhancing the dissolution rate of poorly soluble active ingredients.

Thus, in one aspect, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. an active ingredient or pharmaceutically acceptable salt thereof, wherein said agent has poor water solubility.

As shown in the examples, the inventors have shown that compositions comprising an active ingredient having poor water solubility in amorphous form in a matrix comprising: hydroxypropyl methyl cellulose (HPMC) and/or hydroxypropyl methyl cellulose acetate succinate (HPMC-AS) and polyethylene oxide (PEO); provide sustained release of the active ingredient while enhancing dissolution rate compared to formulations with a crystalline form of the active ingredient. Through in vivo-in vitro correlations, the inventors have shown that these formulations can provide equivalent plasma profiles of the active ingredient using lower doses in a stable manner. Thus, providing a clear advantage due to decreasing the total drug load and increasing efficiency for the administration of poorly soluble drugs.

The inventors have demonstrated that the drug release produced by the compositions of the disclosure can advantageously produce peak concentrations of active ingredients 3 to 6 hours. This provides a balanced release which it advantageous for the dosing of active ingredients throughout the day and before major sleep events.

The specific rate of release achieved by the compositions of the invention is especially advantageous for the delivery of active ingredients that benefit from an administration that matches circadian rhythms, especially nocturnal events associated with circadian rhythms. The inventors have demonstrated that the drug release produced by the compositions of the disclosure can advantageously produce peak concentrations of active ingredients 3 to 6 hours after administration with a safe return to baseline. Thus, allowing for therapeutic concentrations during the night when taken in the evening. This makes the compositions highly attractive for the administration of active ingredients to match their effect to events that naturally oscillate with an increase or a decrease during the night, with a safe return to baseline. An example of such events is regulation of blood pressure during the night.

In another, the present disclosure provides for a method of manufacturing a composition comprising the steps of:
a. preparing a melt uniform dispersion comprising an active ingredient or a pharmaceutically acceptable salt thereof together with HPMC and/or HPMC-AS, and
b. cooling the melt to obtain a solid composition, wherein at least a portion of the active ingredient is in amorphous form.

In yet another aspect, the present disclosure provides for the composition as described herein for use as a medicament.

### DESCRIPTION OF DRAWINGS

FIG. 1: Dissolution rate of 5 mg felodipine tablets where felodipine is either in amorphous or crystalline form.

FIG. 2: Predicted felodipine plasma profile using in vivo-in vitro correlation (IVIVC) at 5 mg dose based on the dissolution rate of tablet comprising amorphous felodipine.

FIG. 3: Predicted felodipine plasma profile using in vivo-in vitro correlation (IVIVC) at 5 mg dose based on the dissolution of the composition comprising crystalline fellodipine and at 0.2 mg dose based on the dissolution rate of composition comprising amorphous felodipine.

### DEFINITIONS

“tmax” is the term used in pharmacokinetics to describe the time at which the Cmax is observed.

“Half-life (t_{1/2})” is a term that refers to the time required for half of the drug in blood to be eliminated.

“Apparent volume of distribution (Vd)” or “Apparent volume of drug distribution” is a parameter calculated from the amount of drug administered and its observed concentration (mass/volume) for an individual with a given body mass.

“Oral bioavailability” refers to the fraction of orally administered drug available in the blood calculated relative to drug concentration after intravenous injection.

“Sustained release” is a term used to refer to a mechanism used in compositions or formulations to provide release of a drug or an active pharmaceutical ingredient from the composition or formulation over an extended period time, as opposed to all at once, or as opposed to an immediate release or a burst release. In some embodiments, as used herein, the terms “sustained release”, “extended release”, “controlled release” and “modified release” are interchangeable to refer to this mechanism.

In some embodiments, as used herein, the terms “composition” and “formulation” are used interchangeably.

An “active ingredient” as used herein refers to any component that provides pharmacological activity or other direct effect in the diagnosis, cure, mitigation, treatment, or prevention of disease, or to affect the structure or any function of the body of man or animals.

“Matrix” as used herein refers to a well-mixed composite of one or more active ingredients with one or more excipients, especially polymers.

The term “about” as used herein to refer to an amount or percentage is to be interpreted as a variation of ±10% with respect the value of the amount or percentage it refers to, such as ±5%.

“Amorphous” as used herein refers to a solid that is amorphous, or that has amorphous parts, or to an active ingredient that is in amorphous form in a solid dispersion. This refers to the solid or the active ingredient in the solid that lacks the long-range order that is characteristic of a crystal. Amorphous materials cannot be defined by a finite unit cell.

“Crystalline” as used herein refers to a solid that is crystalline, or that has crystalline parts, or to an active ingredient that is in crystalline form in a composition. This refers to the solid or the active ingredient having a highly ordered microscopic structure, forming a crystal lattice that extends in all directions.

“Solubility” or “Aqueous solubility” as used herein refers to a property of a substance called solute to dissolve in aqueous medium. The solubility of a substance fundamentally depends on the solvent, temperature, and pressure. The solubility can be measured as the saturation concentration where adding more solute does not increase its concentration in the solution. Active ingredients with poor or low aqueous solubility have a solubility in an aqueous medium below 0.1 g/L.

“Chronotherapy” as used herein refers to the administration of drugs or treatments in a manner that the effects are matched with the natural circadian rhythms.

### DETAILED DESCRIPTION

### A Composition

In one aspect, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. an active ingredient or pharmaceutically acceptable salt thereof, wherein said active ingredient has poor water solubility.

The inventors have shown that compositions comprising an active ingredient and a matrix comprising: hydroxypropyl methyl cellulose (HPMC) and/or hydroxypropyl methyl cellulose acetate succinate (HPMC-AS) and polyethylene oxide (PEO); provide sustained release of the active ingredient with a desirable profile. In addition, the inventors have shown that these compositions can accommodate amorphous forms of active ingredients, thus enhancing the dissolution rate of active ingredients with poor aqueous solubility.

Polyethylene oxide (PEO) is obtained by polymerization of ethylene oxide. It has a similar structure than polyethylene glycol (PEG) which is obtained by condensation of ethylene glycol molecules. In this text, the terms “PEG” and “PEO” are used interchangeably.

HPMC and HPMC-AS are cellulose derivatives wherein some free hydroxyl groups in cellulose have been substituted with hydroxyproyl and methyl groups. In the case of HPMC-AS, further hydroxyl groups are substituted with acetate and succinate groups. Generally, these materials are used as coatings to delay the release of a medicinal compound into the digestive tract.

In one embodiment, the HPMC-AS comprises 4 to 28% by weight succinyl groups and 2 to 16% by weight acetyl groups, for example 8 to 20% by weight succinyl groups and 4 to 12% by weight acteyl groups, for example 14 to 18% by weight succinyl groups and 4 to 9% by weight acetyl groups. In one embodiment, the acetyl content in the HPMC-AS is below 12% by weight, such as below 10% and the succinyl content is above 6% by weight, such as above 8% by weight.

In another embodiment, the composition comprises HPMC and/or HPMC-AS in a total amount of about 1% to 20%, such as about 2% to 20%, such as about 5% to 15%, such as about 6% to 14%, such as about 7% to 13%, such as about 8% to 12%, such as about 9% to 11%, for example about 10%.

Additionally, the inventors have surprisingly shown that compositions comprising HPMC-AS mixed in a matrix, instead of as a coating or encapsulation, provide a sustained release profile with favorable pharmacokinetic properties. Due to its ionic properties, HPMC-AS is able to protect the active ingredient in the gastro-intestinal tract, thus favoring stability in the GI tract allowing improved absorption and bioavailability. In one embodiment, the composition does not comprise HPMC.

Thus, in one embodiment, the composition comprises HPMC-AS in an amount of about 1% to 20% by weight, such as about 2% to 20%, such as about 5% to 15%, such as about 6% to 14%, such as about 7% to 13%, such as about 8% to 12%, such as about 9% to 11%, for example about 10%. In one embodiment, the composition comprises HPMC-AS in an amount of about 1% to 20% by weight, such as about 2% to 20%, such as about 5% to 15%, such as about 6% to 14%, such as about 7% to 13%, such as about 8% to 12%, such as about 9% to 11%, for example about 10% and does not comprise HPMC.

In one embodiment, the composition comprises HPMC-AS in an amount of about 3% to 14% by weight, such as 3% to 4%, such as 4% to 5%, such as 5% to 6%, for example 6% to 7%, such as 7% to 8%, such as 8% to 9%, such as 9% to 10%, such as 10% to 11%, such as 11% to 12%, such as 12% to 13%, such as 13% to 14%. In one embodiment, the composition comprises HPMC-AS in an amount of about 5% to about 14% by weight. In one embodiment, the composition comprises HPMC-AS in an amount of about 8% to about 14% by weight.

In one embodiment, the composition comprises HPMC-AS in an amount of about 3% to 14% by weight, such as 3% to 4%, such as 4% to 5%, such as 5% to 6%, for example 6% to 7%, such as 7% to 8%, such as 8% to 9%, such as 9% to 10%, such as 10% to 11%, such as 11% to 12%, such as 12% to 13%, such as 13% to 14%; and does not comprise HPMC. In one embodiment, the composition comprises HPMC-AS in an amount of about 5% to about 14% by weight and does not comprise HPMC. In one embodiment, the composition comprises HPMC-AS in an amount of about 8% to about 14% by weight and does not comprise HPMC.

In one embodiment, the composition does not comprise HPMC-AS.

In one embodiment, the composition comprises HPMC in an amount of about of about 1% to 20% by weight, such as about 2% to 20%, such as about 5% to 15%, such as about 6% to 14%, such as about 7% to 13%, such as about 8% to 12%, such as about 9% to 11%, for example about 10%.

In one embodiment, the composition comprises HPMC in an amount of about of 5% to 15% by weight, such as 5% to 6%, for example 6% to 7%, such as 7% to 8%, such as 8% to 9%, such as 9% to 10%, such as 10% to 11%, such as 11% to 12%, such as 12% to 13%, such as 13% to 14%, such as 14% to 15%, such as 15% to 16%, such as 16% to 17%, such as 17% to 18%, such as 18% to 19%, such as 19% to 20%.

In one embodiment, the PEO has an average molecular weight between 10 kDa to 2000 kDa, such as 50 kDa to 1000 kDa, such as 100 kDa to 500 kDa, such as 150 kDa to 300 kDa. In one embodiment the PEO has an average molecular weight between 100 kDa and 500 kDa. In one embodiment, the PEO has an average molecular weight between 100 kDa and 400 kDa. In one embodiment, the PEO has an average molecular weight between 100 kDa and 300 kDa. In one embodiment, the PEO has an average molecular weight of about 200 kDa.

In one embodiment, the PEO is present in the composition in an amount of about 80 to 99%, such as about 85% to 95%, such as about 88% to 92%, such as about 90%. In one embodiment, the PEO is present in the composition in an amount of about 80% to 99% by weight. In one embodiment the PEO is present in the composition in an amount of about 83% to 97% by weight. In one embodiment, the PEO is present in the composition in an amount of about 85% to 95% by weight. In one embodiment the PEO is present in the composition in an amount of about 87% to 93% by weight. In one embodiment the PEO is present in the composition in an amount of about 90% by weight.

In one embodiment, the PEO is present in the pharmaceutical composition in an amount of about 85% to 95% by weight, such as 85% to 86%, such as 86% to 87%, such as 87% to 88%, such as 88% to 89%, such as 89% to 90%, such as 90% to 91%, such as 91% to 92%, such as 92% to 93%, such as 93% to 94%, such as 94% to 95% by weight.

In one embodiment, the composition according to the present disclosure is in a solid dosage form, such as a pill, a tablet, a thermoformed unit, or a capsule. In one embodiment, the composition is a single-unit oral dosage form. In one embodiment, the composition according to the present disclosure is in the form of a tablet. For example, the composition is in one embodiment a thermoformed or a compressed tablet.

In one embodiment according to the present disclosure, the active ingredient, the PEO and the HPMC and/or HPMC-AS are in a single matrix. In one embodiment, the composition is a uniform dispersion comprising the active ingredient, the HPMC and/or HPMC-AS and the PEO. “Uniform dispersion” means one wherein the components are evenly or homogeneously distributed through the dispersion.

The compositions according to the present disclosure can accommodate the active ingredient in an amorphous form. An amorphous form can be achieved by preparing a melt state where the active ingredient is homogeneously dispersed with one or more of the matrix components. In the melt state, the active ingredient is partially or totally dissolved in one or more of the matrix components. Thereafter, rapid cooling of the melt prevents the formation of crystalline phases.

Thus, in one embodiment the composition according to the present disclosure comprises the active ingredient, wherein at least a portion of the active ingredient is in an amorphous form. In one embodiment, the composition according to the present disclosure comprises the active ingredient, wherein at least a portion of the active ingredient is an amorphous solid dispersion. For example, the majority of the active ingredient is in an amorphous form or in an amorphous solid dispersion. In one embodiment, at least a portion of the active ingredient is in an amorphous solid dispersion in the matrix. In one embodiment, at least a portion of the active ingredient is in an amorphous dispersion with the HPMC and/or the HPMC-AS and/or the PEO.

The presence of crystalline and amorphous phases can be assessed with different techniques, such as X-ray diffraction (XRD), such as powder X-ray diffraction.

As shown by the data submitted herewith, the inventors have shown that having the active ingredient in an amorphous form enhances the dissolution rate of the active ingredient, while maintaining the sustained release. Thereby providing a solution to achieve increased absorption and bioavailability of poorly soluble drugs with a sustained release profile.

PEO, HPMC and HPMC-AS are regarded as hydrophilic and gel-forming. In water, a matrix made up of any of them or mixtures thereof will experience a series of processes leading to dissolution: water penetration, hydration, disentanglement, gel formation and migration of loose chains into the media.

In cases where the matrix is produced in such a way as to prevent fast penetration of water, the first step, the result is that the dosage unit is gradually eroded only forming a thin release layer on the surface. Such a matrix can be achieved by thermoplastic processes such as injection molding, hot-melt extrusion, calendering or by compression at sufficient pressures due to the high deformability/plasticity and low melting point of, in particular PEO. The object is to prevent water penetration by reducing the occurrence of cracks or inter-particular channels.

The compositions according to the present disclosure release the active ingredient predominantly by erosion. Thus, the active ingredient release closely follows the rate of erosion. The rate of erosion is governed by the specific components in the matrix, namely the PEO and HPMC and/or HPMC-AS as described herein. This mechanism is less prone to variation between different types of active ingredients as opposed to where release is mostly controlled by diffusion. The examples demonstrate that different active ingredients display comparable dissolution profiles.

One aspect of the present disclosure provides for compositions comprising a matrix comprising:
i. an active ingredient or a pharmaceutically acceptable salt thereof as described herein,
ii. HPMC and/or HPMC-AS, and
iii. polyethylene oxide (PEO);
   wherein the weight ratio of PEO (component iii.) to HPMC and/or HPMC-AS (component ii.) is between 8:2 and 9:0.1.

In one embodiment, the PEO, the HPMC, and the HPMC-AS are as described herein. In one embodiment, the weight ratio of PEO to HPMC and/or HPMC-AS is between about 8:1 and about 9.5:1, such as 8.1:1, 8.2:1, 8.3:1, 8.4:1, 8.5:1, 8.6:1, 8.7:1, 8.8:1, 8.9:1, 9.0:1, 9.1:1, 9.2:1, 9.3:1, 9.4:1 or 9.5:1.

In one embodiment, the weight ratio of PEO to HPMC and/or HPMC-AS is 9:1. In one embodiment, the weight ratio of PEO to HPMC and/or HPMC-AS is 8:2. In one embodiment, the weight ratio of PEO to HPMC and/or HPMC-AS 9.5:1. In one embodiment, the weight ratio of PEO to HPMC and/or HPMC-AS 9:0.5. In one embodiment, the weight ratio of PEO to HPMC and/or HPMC-AS 8.5:1. In one embodiment, the weight ratio of PEO to HPMC and/or HPMC-AS 8.6:1.

In one embodiment, the matrix does not comprise HPMC. In one embodiment, the matrix does not comprise HPMC-AS.

In one embodiment, the active ingredient is as described herein in the section “Active pharmaceutical ingredient”.

The inventors have surprisingly shown that the compositions according to the present disclosure do not require the presence of a lubricant. Lubricants are generally required to improve the properties of a powder during processing of formulations, and work by reducing friction. Due to the properties of PEO and the amounts of PEO used, a lubricant is not necessary during processing to prepare the compositions according to the present disclosure. Thus, in one embodiment, the composition according to the present disclosure does not comprise a lubricant. In one embodiment, the composition does not comprise and additional lubricant. For example, the composition does not comprise lubricants selected from: metallic salts of fatty acids, such as divalent salts of fatty acids, for example, magnesium, calcium, or zinc salts of fatty acids; fatty acids, fatty acids esters, or talc.

### Further Polymers

The composition according to the present disclosure may comprise one or more further polymers. The one or more further polymers may be independently selected from the group consisting of ionic, non-ionic, water-insoluble polymers, and water-soluble polymers. In one embodiment, the one or more polymers are one or more water-soluble polymers.

In one embodiment, the one or more further polymers are selected from the group consisting of polysaccharides, acrylates and polysiloxanes and derivatives thereof.

In another embodiment, the one or more further polymers are independently selected from the group consisting of polyethylene oxide glucomannan, galactan, glucan, polygalacturonic acid, polyhydroxyalkanoates, polyxylane, polygalactomannans, rhanogalacturonan, polyxyloglycan, arabinogalactan, starch, alginates, xhanthan gum, carrageenan, agar, dextran, pectins, cellulose, polyvinyl alcohol, polyvinyl butyral, polyvinyl pyrrolidone, methylcellulose, ehtylcellulose, hydroxypropyl cellulose, hydroxypropyl methylcellulose stearate, carboxymethyl cellulose, carbomers, polyacrylic acid, poly(methylacrylic) acid, poly(methylmethacrylate), polyhydroxybutyrate, polyhydroxyvalerate, polyhydroxyphenylvalerate, polylactic acid, polyglycolic acid, a polyacrylic amide, and derivatives or copolymers thereof.

### Active Pharmaceutical Ingredient

The compositions according to the present disclosure provide for a faster dissolution rate of the active ingredient compared to a crystalline form while achieving a specific sustained release. The combination of components in the matrix as described herein accommodates for amorphous forms, thus enhancing the dissolution rate compared to crystalline forms, while providing a specific drug release rate governed by the erosion of the matrix.

In one embodiment, the composition is able to increase the dissolution rate of the active ingredient. In one embodiment, the composition is able to increase the dissolution rate of the active ingredient compared to a composition comprising a crystalline form of the active ingredient. The inventors have shown that compositions prepared comprising an amorphous form of the active ingredient displayed an enhanced dissolution rate compared to compositions wherein the active ingredient is in crystalline form. An increase of the dissolution rate translates also in a faster absorption in the gut. The improved dissolution rate gives a better opportunity for absorption in the gastrointestinal tract. Thus, in one embodiment, the composition is able to increase the absorption rate of the active ingredient in the gut, such as in the intestines.

In one embodiment, the composition is able to increase the oral bioavailability of the active ingredient. In one embodiment, the composition is able to increase the oral bioavailability of the active ingredient compared to a composition comprising a crystalline form of the active ingredient.

The compositions according to the present disclosure thus, may be particularly useful for active ingredients having low solubility in aqueous media. For example for active ingredients, or pharmaceutically acceptable salts thereof, that fall into class II or class IV of the Biopharmaceutics Classification System (BCS)². Class II corresponds to active ingredients having low solubility and high permeability, while Class IV corresponds to active ingredients having low solubility and low permeability.

The BCS is a system that classifies active ingredients according to their solubility and permeability. The solubility criteria under the BCS defines a high solubility class boundary as substances for which the highest marketed dose is soluble in 250 mL of water over the pH range of 1-7.5 at 37° C.

According to the U.S Food and Drug Administration (FDA) implemented BCS guidance², solubility can be determined as equilibrium solubility using a shake-flask technique or alternative methods, over suitable timeframes to reach equilibrium and confirming stability of the drug in the media used for testing.

The permeability class boundaries in the BCS are based on the extent of absorption of an active ingredient in humans.

According to the U.S Food and Drug Administration (FDA) implemented BCS guidance², the assessment of permeability should preferentially be based on the extent of absorption derived from human pharmacokinetic studies, e.g., absolute bioavailability or mass balance; permeability can be also assessed by validated and standardized in vitro methods using Caco-2 cells.

High permeability can be concluded when the absolute bioavailability is >85%. The BCS class for permeability may be determined by measuring human effective permeability rates (P_{eff}) across the jejunal membrane in humans; or by measuring apparent permeability rates (Pₐₚₚ) across in vitro epithelial cell monolayers, such as human intestinal cell line Caco-2 compared to controls of known permeability (see Table 2 in reference²) and accounting for transport of the drug substance².

Thus, according to the above, a person of skill in the art will be aware on how to determine BCS classification of an active ingredient.

In one embodiment, the active ingredient is an active ingredient that falls into the criteria of Class II or Class IV of the BCS classification. In one embodiment, the active ingredient is an active ingredient that falls into the criteria of Class II of the BCS classification. In one embodiment, the active ingredient is an active ingredient that falls into the criteria of Class IV of the BCS classification. In one embodiment, the active ingredient's highest intended dose is not soluble in 250 mL of water over the pH range of 1.0-7.5 at 37° C.

The composition according to the present disclosure comprises and active ingredient having low solubility in aqueous media. For example, active ingredients having a solubility in aqueous media, such as water or buffered saline, below 0.1 g/L, such as below 0.08 g/L, such as below 0.06 g/L, such as below 0.04 g/L, such as below 0.02 g/L, such as below 0.01 g/L.

Thus in one embodiment, the active ingredient has a solubility in an aqueous medium below 0.1 g/L. In one embodiment, the active ingredient has a solubility in water or in buffered saline below 0.1 g/L.

In one embodiment, the active ingredient has a high permeability according to the BCS classification, for example the active ingredient has a bioavailability higher than 85% as assessed by BCS compatible methods described above. In one embodiment, the active ingredient has a bioavailability lower than 85%, such as lower than 70%, lower than 60%, or lower than 55% as assessed by BCS compatible methods described above. In one embodiment, the active ingredient has a bioavailability from 75% to 85%, or from 65% to 75%, or from 55% to 65% as assessed by BCS compatible methods described above.

In one embodiment, the active ingredient is a substance having poor solubility according to the BCS classification. In one embodiment, the active ingredient is an active ingredient having poor solubility according to the BCS classification system selected from any one of the group consisting of:
afabicin, afabicin desphosphono, cobicistat, pralsetinib, piroxicam, tamoxifen glyburide, etodolac, tinidazole, bezafibrate, efavirenz, nevirapine, ergometrine, orismilast, nalmefene, asalhydromorphone, firategrast, lamotrigine, silibinin, hydrochlorothiazide, felodipine, posaconzaole, terbinafine, olorifim, cariprazine, tibolone, sertindole, daclatasvir, risperidone, pitavastatin, lovastatin, estradiol, raloxifene, mercaptopurine, atuliflapon, guanfacine, espirolactone, enzalutamide, firibastat, abrocitinib, glimpiride, ketoconazole, 1-methylnicotinamide, ibrutinib, rivaroxaban, safinamide, budesonide, ralinepag, valsartan, rimegepant, magnesium hydroxide, bicalutamide, vericiciguat, buphenine, bromocriptine, daridorexant, dipraglurant, dexlansoprazole, esomeprazole, sotagliflozin, duloxetine, belzutifan, sibutramine, loperamide, asciminib, basimglurant, deucravacitinib, fenofibrate, omeprazole, buprenorphine, rolapitant, lysine, finerenone, telmisartan, ansornitinib, mirtazapine, methimazole, temazepam, tadalafil, vardenafil, streptokinase, clopidogrel, nolatrexed, olanzapine, vilaprisan, cefuroxime, vapreotide, amprenavir, mefenamic acid, chlorzoxazone, drotaverine, butylscopolamine, danuglipron, palbociclib isethionat, gepotidacin, fostemsavir, calcium carbonate, pelubiprofen, zuclopentixol, zileuton, oxaprozin methylphenidate, phenazopyridine, flavoxate, methoxsalen, naproxen, lamotrigine, clenbuterol, hydroxyzine, loracarbef, ezogabine, phenytoin, racecadotril, crizotinib, griseofulvin, terbinafine, leniolisib, paliperidone, pemafibrate, nicardipine, amiodarone, carvedilol, methenamine, dicloxacillin, ketoconazole, tianeptine, onapristone, nitrofurantoin, loxapine, diacerein, bosentan, carbamazepine, omecamtiv mecarbil, oxacarbazepine, panobinostat, CE 224535 (CAS 724424-43-5), diclofenac, naltrexone, nicergoline, methocarbamol, metaxalone, chlorzoxazone, tizanidine, prednisolone, dipyridamole, mycophenolic acid, mycophenolate, felbamate, diosamin, sucralfate, gemfibrozil, carisoproldol, aplindore, cyclosporine, clonazepam, orlistat, sildenafil, lesinurad, cetylpyridinium, benzodiazepine, trimebutine, testosterone, betamethasone, gabapentin enacarbil, gabapentin, latanoprost, prochlorperazine, celecoxib, aceclofenac, nitazoxanide, dolutegravir, sulpiride, venetoclax, quinapril, cinacalcet, abiraterone, allopurinol, rifampicin, etravirine, acetazolamide, pyrantel, aripiprazole, sulfamethoxazole, venetoclax, ranolazine, folic acid, furosemide, azathioprine, chlorthalidone, adrulipase alfa, mifepristone, camlipixant, cethromycin, eritromycin, mesalazine, ritonavir, cisapride, apremilast, tannic acid, tolvaptan, canagliflozin, digoxin, cisplatin, ticagrelor, rifaximin, aprazolam, ketamine, albendazole, axitinib, metformin, chloropyramine, cinnarizine, clarithromycin, danazol, dextromethorphan, dimeticone, dronabinol, tramadol, dyclonine, aluminiumhydroxide, indomethacin, ketoprofen, ketorolac, letermovir, lornoxicam, maribavir, mebendazole, nicerogline, oxaprozin, oxcarbazepine, pancreatin, pemigatinib, pinaverium, pioglitazone, scopolamine, and selexipag, or a pharmaceutically acceptable salt thereof.

The composition of the present disclosure may be particularly useful for formulation of active ingredients falling into BCS Class II, as increasing the dissolution rate will enhance the rate of absorption due to the high permeability of these active ingredients. Thus, in one embodiment, the active ingredient is a BCS Class II substance. In one embodiment, the active ingredient is a BCS Class II substance selected from any one of the group consisting of:
afabicin, afabicin desphosphono, cobicistat, pralsetinib, piroxicam, tamoxifen glyburide, etodolac, tinidazole, bezafibrate, efavirenz, nevirapine, ergometrine, orismilast, nalmefene, asalhydromorphone, firategrast, lamotrigine, silibinin, hydrochlorothiazide, felodipine, posaconzaole, terbinafine, olorifim, cariprazine, tibolone, sertindole, daclatasvir, risperidone, pitavastatin, lovastatin, estradiol, raloxifene, mercaptopurine, atuliflapon, guanfacine, espirolactone, enzalutamide, firibastat, abrocitinib, glimpiride, ketoconazole, 1-methylnicotinamide, ibrutinib, rivaroxaban, safinamide, budesonide, ralinepag, valsartan, rimegepant, magnesium hydroxide, bicalutamide, vericiciguat, buphenine, bromocriptine, daridorexant, dipraglurant, dexlansoprazole, esomeprazole, sotagliflozin, duloxetine, belzutifan, sibutramine, loperamide, asciminib, basimglurant, deucravacitinib, fenofibrate, omeprazole, buprenorphine, rolapitant, lysine, finerenone, telmisartan, ansornitinib, mirtazapine, methimazole, temazepam, tadalafil, vardenafil, streptokinase, clopidogrel, nolatrexed, olanzapine, vilaprisan, cefuroxime, vapreotide, amprenavir, mefenamic acid, chlorzoxazone, drotaverine, butylscopolamine, danuglipron, palbociclib Isethionat, gepotidacin fostemsavir, calcium carbonate, pelubiprofen, zuclopentixol, zileuton, oxaprozin methylphenidate, phenazopyridine, flavoxate, methoxsalen, naproxen, lamotrigine, clenbuterol, hydroxyzine, loracarbef, ezogabine, phenytoin, racecadotril, crizotinib, griseofulvin, terbinafine, leniolisib, paliperidone, pemafibrate, nicardipine, amiodarone, carvedilol, methenamine, dicloxacillin, ketoconazole, tianeptine, onapristone, nitrofurantoin, loxapine, diacerein, bosentan, carbamazepine, omecamtiv mecarbil, oxacarbazepine, panobinostat, CE-224535 (CAS 724424-43-5), diclofenac, naltrexone, nicergoline, methocarbamol, metaxalone, chlorzoxazone, tizanidine, prednisolone, dipyridamole, mycophenolic acid, mycophenolate, felbamate, diosamin, sucralfate, gemfibrozil, carisoprodol, aplindore, cyclosporine, clonazepam, orlistat, sildenafil, lesinurad, cetylpyridinium, benzodiazepine, trimebutine, testosterone, betamethasone, gabapentin enacarbil, gabapentin, latanoprost, prochlorperazine, celecoxib, aceclofenac, nitazoxanide, and dolutegravir, albendazole, axitinib, metformin, chloropyramine, cinnarizine, clarithromycin, danazol, dextromethorphan, dimeticone, dronabinol, tramadol, dyclonine, aluminiumhydroxide, indomethacin, ketoprofen, ketorolac, letermovir, lornoxicam, maribavir, mebendazole, nicerogline, oxaprozin, oxcarbazepine, pancreatin, pemigatinib, pinaverium, pioglitazone, scopolamine, and selexipag, or a pharmaceutically acceptable salt thereof.

The composition of the present disclosure may be particularly useful for formulation of active ingredients falling into BCS Class IV, because they enhance dissolution rate and the matrix protects the active ingredient in the GI-tract thus favoring absorption. Thus, in one embodiment, the active ingredient is a BCS Class IV substance. In one embodiment, the active ingredient is a BCS Class IV substance selected from any one of the group consisting of:
sulpiride, venetoclax, quinapril, cinacalcet, abiraterone, allopurinol, rifampicin, etravirine, acetazolamide, pyrantel, aripiprazole, sulfamethoxazole, venetoclax, ranolazine, folic acid, furosemide, azathioprine, chlorthalidone, adrulipase alfa, mifepristone, camlipixant, cethromycin, eritromycin, mesalazine, ritonavir, cisapride, apremilast, tannic acid, tolvaptan, canagliflozin, digoxin, cisplatin, ticagrelor, rifaximin, aprazolam, and ketamine, or a pharmaceutically acceptable salt thereof. In one embodiment, the active ingredient is selected from any one of the group consisting of: sulpiride, venetoclax, quinapril, cinacalcet, abiraterone, allopurinol, rifampicin, etravirine, acetazolamide, pyrantel, aripiprazole, sulfamethoxazole, venetoclax, ranolazine, folic acid, furosemide, azathioprine, chlorthalidone, adrulipase alfa, and mifepristone, or a pharmaceutically acceptable salt thereof.

In one embodiment, the active ingredient is selected from any one of the group consisting of: camlipixant, cethromycin, eritromycin, mesalazine, ritonavir, cisapride, apremilast, tannic acid, tolvaptan, canagliflozin, digoxin, cisplatin, ticagrelor, rifaximin, aprazolam, and ketamine, or a pharmaceutically acceptable salt thereof.

In one embodiment, the active ingredient is selected from any one of the group consisting of: afabicin, afabicin desphosphono, cobicistat, pralsetinib, piroxicam, tamoxifen glyburide, etodolac, tinidazole, bezafibrate, efavirenz, nevirapine, ergometrine, orismilast, nalmefene, asalhydromorphone, firategrast, lamotrigine, silibinin, hydrochlorothiazide, felodipine, posaconzaole, terbinafine, olorifim, cariprazine, tibolone, sertindole, daclatasvir, risperidone, pitavastatin, lovastatin, estradiol, raloxifene, mercaptopurine, atuliflapon, guanfacine, espirolactone, enzalutamide, firibastat, abrocitinib, glimpiride, ketoconazole, 1-methylnicotinamide, ibrutinib, rivaroxaban, safinamide, budesonide, ralinepag, valsartan, rimegepant, magnesium hydroxide, bicalutamide, vericiciguat, buphenine, bromocriptine, daridorexant, dipraglurant, dexlansoprazole, esomeprazole, sotagliflozin, duloxetine, belzutifan, sibutramine, loperamide, asciminib, basimglurant, deucravacitinib, fenofibrate, omeprazole, buprenorphine, rolapitant, lysine, finerenone, telmisartan, ansornitinib, mirtazapine, methimazole, temazepam, tadalafil, vardenafil, streptokinase, clopidogrel, nolatrexed, olanzapine, and vilaprisan, or a pharmaceutically acceptable salt thereof.

In one embodiment, the active ingredient is selected from any one of the group consisting of: cefuroxime, vapreotide, amprenavir, mefenamic acid, chlorzoxazone, drotaverine, butylscopolamine, danuglipron, palbociclib Isethionat, gepotidacin fostemsavir, calcium carbonate, pelubiprofen, zuclopentixol, zileuton, oxaprozin methylphenidate, phenazopyridine, flavoxate, methoxsalen, naproxen, lamotrigine, clenbuterol, hydroxyzine, loracarbef, ezogabine, phenytoin, racecadotril, crizotinib, griseofulvin, terbinafine, leniolisib, paliperidone, pemafibrate, nicardipine, amiodarone, carvedilol, methenamine, dicloxacillin, ketoconazole, tianeptine, onapristone, nitrofurantoin, loxapine, diacerein, bosentan, carbamazepine, omecamtiv mecarbil, oxacarbazepine, panobinostat, CE-224535 (CAS 724424-43-5), diclofenac, naltrexone, nicergoline, methocarbamol, metaxalone, chlorzoxazone, tizanidine, prednisolone, dipyridamole, mycophenolic acid, mycophenolate, felbamate, diosamin, sucralfate, gemfibrozil, carisoprodol, aplindore, cyclosporine, clonazepam, orlistat, sildenafil, lesinurad, cetylpyridinium, benzodiazepine, trimebutine, testosterone, betamethasone, gabapentin enacarbil, gabapentin, latanoprost, prochlorperazine, celecoxib, aceclofenac, nitazoxanide, and dolutegravir, or a pharmaceutically acceptable salt thereof.

In one embodiment, the composition according to the present disclosure comprises the active ingredient in an amount equal or lower than 10% by weight, such as lower than 9%, 8%, 7%, 6% or 5% by weight. In one embodiment, the composition comprises the active ingredient in an amount equal or lower to 5% by weight.

In one embodiment, the composition according to the present disclosure comprises the active ingredient in an amount equal or lower than 200 mg, such as between 0.001 mg and 200 mg, such as between 0.001 mg and 100 mg, such as between 0.01 mg and 100 mg, such as between 0.05 and 100 mg, such as between 1 mg and 100 mg.

In one embodiment, the composition is able to increase tₘₐₓ of the active ingredient, as compared to an equivalent amount of the active ingredient administered as an immediate-release formulation.

In one embodiment, the tₘₐₓ of the active ingredient is increased by at least about 1 hour, such as at least about 2 hours, for example at least about 3 hours, such as at least about 4 hours as compared to an equivalent amount of the active ingredient administered as an immediate release formulation.

In one embodiment, upon oral administration, the composition provides for tₘₐₓ of 3 to 6 hours, such as a tₘₐₓ of 3.0, 3.5, 4.0, 4.5, 5.0, 5.5, or 6.0 hours. In one embodiment, the composition provides for tₘₐₓ 3.0 to 4.0 hours. In one embodiment, the composition provides for tₘₐₓ 4.0 to 5.0 hours. In one embodiment, the composition provides for tₘₐₓ 5.0 to 6.0 hours.

The composition of the present disclosure provides for an increase in the dissolution rate of poorly soluble active ingredients and an increase in bioavailability, while simultaneously providing a specific drug release rate that is advantageous for chronotherapy, for example in the delivery of active ingredients that benefit from administration that matches circadian rhythms, especially nocturnal events associated with circadian rhythms.

As shown herein, the specific drug release rate provided by the compositions of the present disclosure allows achieving a peak concentration with a tₘₐₓ of between 3 to 6 hours with a safe return to baseline in humans as predicted by in vivo-in vitro correlations (IVIVC). This is specifically advantageous for the administration of active ingredients that regulate events that increase or decrease during the night, for example, blood pressure.

Thus, in one embodiment, the active ingredient is an active ingredient that regulates blood pressure. In one embodiment, the active ingredient is an active ingredient that regulates blood pressure having low solubility in aqueous media as described herein, for example having low solubility according to the BCS classification system. In one embodiment, the active ingredient is an anti-hypertensive, such as an anti-hypertensive having low solubility in aqueous media as described herein. In one embodiment, the anti-hypertensive is selected from the group consisting of: felodipine, nicardipine, valsartan, and telmisartan, or a pharmaceutically acceptable salt thereof.

In one embodiment, the active ingredient is an active ingredient that regulates cardiac events or cardiovascular events. In one embodiment, the active ingredient is an active ingredient that regulates cardiac events or cardiovascular events, that benefits from chronotherapy. In one embodiment, the active ingredient is an active ingredient that regulates cardiac events or cardiovascular events, that benefits from chronotherapy selected from the group consisting of: felodipine, nicardipine, valsartan, telmisartan, chlorathalidone, digoxin, and temazepam, or a pharmaceutically acceptable salt thereof.

In one embodiment, the active ingredient is felodipine.

In one embodiment the active ingredient is selected from the group consisting of: felodipine (CAS number 72509-76-3), naproxen (CAS number 22204-53-1) or intraconazole (CAS number 84625-61-6), or a pharmaceutically acceptable salt thereof. In one embodiment, the active ingredient is felodipine (CAS number 72509-76-3) or a pharmaceutically acceptable salt thereof. In one embodiment, the active ingredient is naproxen (CAS number 22204-53-1) or a pharmaceutically acceptable salt thereof. In one embodiment, the active ingredient is intraconazole (CAS number 84625-61-6) or a pharmaceutically acceptable salt thereof.

In one embodiment, the active ingredient is dotraverine, or a pharmaceutically acceptable salt thereof, such as dotraverine hydrochloride (CAS number 985-12-6).

In one embodiment, the active ingredient is butylscopolamine, or a pharmaceutically acceptable salt thereof, such as butylscopolamine bromide (CAS number 149-64-4).

In one embodiment, the active ingredient is calcium carbonate (CAS number 471-34-1).

In one embodiment, the active ingredient is cethromycin (CAS number 205110-48-1), or a pharmaceutically acceptable salt thereof.

In one embodiment, the active ingredient is pelubiprofen (CAS number 69956-77-0), or a pharmaceutically acceptable salt thereof.

In one embodiment, the active ingredient is zileuton (CAS number 111406-87-2), or a pharmaceutically acceptable salt thereof.

In one embodiment, the active ingredient is flavoxate (CAS number 15301-69-6), or a pharmaceutically acceptable salt thereof.

In one embodiment, the active ingredient is phenazopyridine (CAS number 94-78-0), or a pharmaceutically acceptable salt thereof.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight as described herein, and
b. polyethylene oxide (PEO) in an amount from 80 to 99.9% by weight as described herein, and
c. an active ingredient having low water solubility as described herein, such as an active ingredient that is a BCS class II or BCS class IV substance, or a pharmaceutically acceptable salt thereof,
wherein at least a portion of the active ingredient is in an amorphous solid dispersion.

In one embodiment, the present disclosure provides for a composition comprising:
a. hydroxypropyl methyl cellulose acetate succinate (HPMC-AS) in a total amount of 0.1% to 20% by weight, such as from 5% to 15% by weight as described herein, and
b. polyethylene oxide (PEO) in an amount from 80 to 99.9% by weight as described herein, and
c. an active ingredient having low water solubility as described herein, such as an active ingredient that is a BCS class II or BCS class IV substance, or a pharmaceutically acceptable salt thereof,
wherein at least a portion of the active ingredient is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight as described herein, and
b. polyethylene oxide (PEO) in an amount from 80 to 99.9% by weight as described herein, and
c. an active ingredient that is an anti-hypertensive as described herein, the active ingredient having low water solubility as described herein, such as an active ingredient that is a BCS class II or BCS class IV substance, or a pharmaceutically acceptable salt thereof,
wherein at least a portion of the active ingredient is in an amorphous solid dispersion.

In one embodiment, the present disclosure provides for a composition comprising:
a. hydroxypropyl methyl cellulose acetate succinate (HPMC-AS) in a total amount of 0.1% to 20% by weight, such as from 5% to 15% by weight as described herein, and
b. polyethylene oxide (PEO) in an amount from 80 to 99.9% by weight as described herein, and
c. an active ingredient that is an anti-hypertensive as described herein, the active ingredient having low water solubility as described herein, such as an active ingredient that is a BCS class II or BCS class IV substance, or a pharmaceutically acceptable salt thereof,
wherein at least a portion of the active ingredient is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight as described herein, and
b. polyethylene oxide (PEO) in an amount from 80 to 99.9% by weight as described herein, and
c. felodipine or a pharmaceutically acceptable salt thereof,
   wherein at least a portion of the felodipine is in an amorphous solid dispersion.

In one embodiment, the present disclosure provides for a composition comprising:
a. hydroxypropyl methyl cellulose acetate succinate (HPMC-AS) in a total amount of 0.1% to 20% by weight, such as from 5% to 15% by weight as described herein, and
b. polyethylene oxide (PEO) in an amount from 80 to 99.9% by weight as described herein, and
c. felodipine or a pharmaceutically acceptable salt thereof,
wherein at least a portion of the felodipine is in an amorphous solid dispersion.

In one embodiment, the present disclosure provides for a composition comprising a matrix comprising:
i. felodipine or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least wherein at least a portion of the felodipine is in an amorphous solid dispersion in the matrix.

In one embodiment, the composition comprises felodipine or a pharmaceutically acceptable salt thereof in an amount between 0.05 mg and 10 mg, such as between 0.05 mg and 0.1 mg, such as between 0.1 mg and 1 mg, such as between 1 mg and 2 mg, such as between 2 mg and 3 mg, such as between 3 mg and 4 mg, such as between 4 mg and 5 mg, such as between 5 mg and 6 mg, such as between 6 mg and 7 mg, such as between 7 mg and 8 mg, such as between 8 mg and 9 mg, such as between 9 mg and 10 mg.

In one embodiment, the composition comprises felodipine or a pharmaceutically acceptable salt thereof between 0.05 and 2 mg, such as for example 0.1 mg, 0.2 mg, 0.4 mg, 0.6 mg, 0.8 mg, 1 mg, 1.2 mg, 1.4 mg, 1.6 mg, 1.8 mg or 2 mg of felodipine or a pharmaceutically acceptable salt thereof.

In one embodiment, the composition according to the present disclosure is in a solid dosage form comprising 0.05 mg to 10 mg of felodipine, such as 0.05 mg to 5 mg of felodipine, such as for example 0.05 mg to 2 mg of felodipine.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. naproxen or a pharmaceutically acceptable salt thereof,
wherein at least a portion of the naproxen is in an amorphous solid dispersion.

In one embodiment, the present disclosure provides for a composition comprising a matrix comprising:
i. naproxen or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least wherein at least a portion of the naproxen is in an amorphous solid dispersion in the matrix.

In one embodiment, the composition comprises naproxen or a pharmaceutically acceptable salt thereof in an amount between 1 mg and 1500 mg.

In one embodiment, the composition comprises naproxen or a pharmaceutically acceptable salt thereof in an amount between 1 mg and 300 mg.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. itraconazole or a pharmaceutically acceptable salt thereof,
wherein at least a portion of the itraconazole is in an amorphous solid dispersion.

In one embodiment, the present disclosure provides for a composition comprising a matrix comprising:
i. itraconzole, or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least a portion of the itraconazole is in an amorphous solid dispersion in the matrix.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. drotaverine, or a pharmaceutically acceptable salt thereof,
wherein at least a portion of the dotraverine is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. drotaverine, or a pharmaceutically acceptable salt thereof,
wherein at least a portion of the dotraverine is in an amorphous solid dispersion.

In one embodiment, the present disclosure provides for a composition comprising a matrix comprising:
i. dotraverine, or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least wherein at least a portion of the dotraverine is in an amorphous solid dispersion in the matrix.

In one embodiment, the composition comprises drotaverine or a pharmaceutically acceptable salt thereof in an amount between 1 mg and 80 mg.

In one embodiment, the composition comprises dotraverine or a pharmaceutically acceptable salt thereof in an amount between 1 mg and 20 mg.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. butylscopolamine, or a pharmaceutically acceptable salt thereof,
wherein at least a portion of the butylscopolamine is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. butylscopolamine, or a pharmaceutically acceptable salt thereof,
wherein at least a portion of the butylscopolamine is in an amorphous solid dispersion.

In one embodiment, the present disclosure provides for a composition comprising a matrix comprising:
i. butylscopolamine, or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least a portion of the butylscopolamine is in an amorphous solid dispersion in the matrix.

In one embodiment, the composition comprises butylscopolamine or a pharmaceutically acceptable salt thereof in an amount between 1 mg and 20 mg.

In one embodiment, the composition comprises butylscopolamine or a pharmaceutically acceptable salt thereof in an amount between 1 mg and 5 mg.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. calcium carbonate,
wherein at least a portion of the calcium carbonate is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. calcium carbonate,
wherein at least a portion of the calcium carbonate is in an amorphous solid dispersion.

In one embodiment, the present disclosure provides for a composition comprising a matrix comprising:
i. calcium carbonate, or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least a portion of the calcium carbonate is in an amorphous solid dispersion in the matrix.

In one embodiment, the composition comprises calcium carbonate, or a pharmaceutically acceptable salt thereof in an amount between 1 mg and 7.5 g.

In one embodiment, the composition comprises calcium carbonate or a pharmaceutically acceptable salt thereof in an amount between 1 mg and 1.5 g.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. cethromycin, or a pharmaceutically acceptable salt thereof,
wherein at least a portion of the cethromycin is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. cethromycin, or a pharmaceutically acceptable salt thereof,
wherein at least a portion of the cethromycin is in an amorphous solid dispersion.

In one embodiment, the present disclosure provides for a composition comprising a matrix comprising:
i. cethromycin, or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least a portion of the cethromycin is in an amorphous solid dispersion in the matrix.

In one embodiment, the composition comprises cethromycin or a pharmaceutically acceptable salt thereof in an amount between 1 mg and 300 mg.

In one embodiment, the composition comprises cethromycin or a pharmaceutically acceptable salt thereof in an amount between 1 mg and 60 mg.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. pelubiprofen, or a pharmaceutically acceptable salt thereof,
wherein at least a portion of the pelubiprofen is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. pelubiprofen, or a pharmaceutically acceptable salt thereof,
wherein at least a portion of the pelubiprofen is in an amorphous solid dispersion.

In one embodiment, the present disclosure provides for a composition comprising a matrix comprising:
i. pelubiprofen, or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least a portion of the pelubiprofen is in an amorphous solid dispersion in the matrix.

In one embodiment, the composition comprises pelubiprofen or a pharmaceutically acceptable salt thereof in an amount between 1 mg and 120 mg.

In one embodiment, the composition comprises pelubiprofen or a pharmaceutically acceptable salt thereof in an amount between 1 mg and 24 mg.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. zileuton, or a pharmaceutically acceptable salt thereof,
wherein at least a portion of the zileuton is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. zileuton or a pharmaceutically acceptable salt thereof,
wherein at least a portion of the zileuton is in an amorphous solid dispersion.

In one embodiment, the present disclosure provides for a composition comprising a matrix comprising:
i. zileuton or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least a portion of the zileuton is in an amorphous solid dispersion in the matrix.

In one embodiment, the composition comprises zileuton or a pharmaceutically acceptable salt thereof in an amount between 1 mg and 600 mg.

In one embodiment, the composition comprises zileuton or a pharmaceutically acceptable salt thereof in an amount between 1 mg and 160 mg.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. flavoxate, or a pharmaceutically acceptable salt thereof,
wherein at least a portion of the flavoxate is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. flavoxate or a pharmaceutically acceptable salt thereof,
wherein at least a portion of the flavoxate is in an amorphous solid dispersion.

In one embodiment, the present disclosure provides for a composition comprising a matrix comprising:
i. flavoxate or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least a portion of the flavoxate is in an amorphous solid dispersion in the matrix.

In one embodiment, the composition comprises flavoxate or a pharmaceutically acceptable salt thereof in an amount between 1 mg and 200 mg.

In one embodiment, the composition comprises flavoxate or a pharmaceutically acceptable salt thereof in an amount between 1 mg and 40 mg.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. phenazopyridine, or a pharmaceutically acceptable salt thereof,
wherein at least a portion of the phenazopyridine is in an amorphous solid dispersion.

In one embodiment, the present disclosure provides for a composition comprising:
a. hydroxypropyl methyl cellulose acetate succinate (HPMC-AS) and/or hydroxypropyl methyl cellulose in a total amount of 0.1% to 20% by weight, and
b. polyethylene oxide (PEO) in an amount from 80 to 99.9% by weight, and
c. phenazopyridine or a pharmaceutically acceptable salt thereof,
wherein at least a portion of the phenazopyridine is in an amorphous solid dispersion.

In one embodiment, the present disclosure provides for a composition comprising a matrix comprising:
i. phenazopyridine or a pharmaceutically acceptable salt thereof,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1; and
wherein at least a portion of the phenazopyridine is in an amorphous solid dispersion in the matrix.

In one embodiment, the composition comprises phenazopyridine or a pharmaceutically acceptable salt thereof in an amount between 1 mg and 200 mg.

In one embodiment, the composition comprises phenazopyridine or a pharmaceutically acceptable salt thereof in an amount between 1 mg and 40 mg.

In one embodiment, the composition according to the present disclosure is to be administered between 18 h and 00 h, such as between 20 h and 00 h, for example at 20 h, 21 h, 22 h, 23 h or 00 h.

In one embodiment, the composition according to the present disclosure is to be administered during dinner or after dinner, such as within 3 hours after dinner, such as within 2 hours after dinner, such as within 1 hour after dinner, such as within 30 minutes after dinner.

In one embodiment, the composition according to the present disclosure is to be administered prior to a major sleep episode such as within 3 hours before a major sleep episode, such as within 2 hours before a major sleep episode, for example within 1 hour before a major sleep episode, such as within 30 minutes before a major sleep episode.

In one embodiment, the composition according to the present disclosure is to be administered on an empty stomach.

In one embodiment, the composition according to the present disclosure is to be administered once daily.

In one embodiment, the composition according to the present disclosure is to be administered more than once daily, for example, 2 times daily, 3 times daily, or 4 times daily.

### Method of Manufacturing

In one aspect, the present disclosure provides for a method of manufacturing a composition comprising the steps of:
a. preparing a melt uniform dispersion comprising an active ingredient or a pharmaceutically acceptable salt thereof together with HPMC and/or HPMC-AS, and
b. cooling the melt to obtain a solid composition, wherein at least a portion of the active ingredient is in amorphous form.

In one embodiment, step a. further comprises PEO. In one embodiment, step a. involves heating the mixture to a temperature wherein the active ingredient becomes soluble in the HPMC and/or HPMC-AS. In one embodiment, step a. involves heating the mixture to a temperature wherein the active ingredient becomes soluble in the mixture of HPMC and/or HPMC-AS with PEO. In one embodiment, step a. involves heating the mixture to a temperature wherein the active ingredient becomes amorphous.

Preparing a melt wherein the active ingredient becomes soluble in the matrix ensures that an amorphous form of the active ingredient can be produced.

In one embodiment, the method further comprises a step of combining the composition in b. with PEO.

In one embodiment, the method further comprises a step of compressing the composition into a tablet.

In one embodiment, the active ingredient in step a. is felodipine or a pharmaceutically acceptable salt thereof.

In one embodiment, the active ingredient, the HPMC, the HPMC-AS and the PEO are as described elsewhere herein.

In one embodiment, the melt in step a. is prepared by heating to a temperature above the melting temperature (Tₘ) of one of the matrix components. For example, above Tₘ of HPMC, or above Tₘ of HPMC-AS. In one embodiment, the melt in step a. is prepared by heating the active ingredient and HPMC-AS above 138° C., such as from 140° C. to 190° C., for example from 140° C. to 180° C., for example from 140° C. to 170° C., such as 140° C., 150° C., or 160° C.

The temperature at which an active ingredient changes state and becomes amorphous can be measured by for example, calorimetry techniques such as differential scanning calorimetry (DSC), other methods will be known to someone of skill in the art.

The processes and methodologies to achieve compositions according to the present disclosure are well known to a person of skill in the art. For example, methodologies to implement the method herein described or to prepare the composition described herein include but are not limited to: dry granulation such as achieved by roller compaction, calendering, slugging or pneumatic dry granulation; compression moulding, such as vacuum compression moulding (VCM); direct compression (DCT); hot melt extrusion (HME); ultrasound-assisted compaction; or wet granulation.

### Medical Use

In one aspect, the present disclosure provides for the composition as described herein for use as a medicament.

In one aspect, the present disclosure provides for the use of the composition as described herein in the manufacture of a medicament.

In one aspect, the present disclosure provides a method of treatment of a disease, said method comprising administering the composition as described herein to a subject in need thereof.

As shown herein, the composition of the present disclosure provides for a drug release that is advantageous for chronotherapy of active ingredients regulating cardiac events, and blood pressure, because it allows achieving peak concentrations of the active ingredient during the night, matching natural circadian rhythms. In one aspect, the present disclosure provides for the composition as described herein for use in the treatment, prevention, or alleviation of a hypertensive disease, angina pectoris, or acute myocardial infarction.

In one embodiment, the hypertensive disease is selected from hypertension, essential hypertension, hypertensive heart disease, hypertensive crisis, or secondary hypertension.

In one embodiment, the myocardial infarction is selected from acute myocardial infarction or subsequent myocardial infarction.

In one embodiment, the angina pectoris is unstable or stable angina, angina syndrome or ischaemic chest pain.

In one aspect, the present disclosure provides for the use of the composition as described herein in the manufacture of a medicament for the treatment, prevention, or alleviation of an hypertensive disease, angina pectoris, or acute myocardial infarction.

In one aspect, the present disclosure provides for the composition as described herein for use in the treatment, prevention, or alleviation of pain such as pain associated with the common cold, headache, toothache, muscular aches or backache; inflammatory diseases, such as rheumatoid arthritis, osteoarthritis, ankylosing spondylitis, juvenile arthritis, tendonitis, bursitis or acute gout; dysmenorrhea or fever.

In one aspect, the present disclosure provides for the composition as described herein for use in the treatment, prevention, or alleviation of a fungal infection, such as blastomycosis, histoplasmosis, aspergillosis, or onychomycosis.

### Items

1. A composition comprising:
   a. hydroxypropyl methyl cellulose acetate succinate (HPMC-AS) and/or hydroxypropyl methyl cellulose in a total amount of 0.1% to 20% by weight, and
   b. polyethylene oxide (PEO) in an amount from 80 to 99.9% by weight, and
   c. an active ingredient or a pharmaceutically acceptable salt thereof, wherein at least a portion of the active ingredient or pharmaceutically acceptable salt thereof is in an amorphous form.
2. The composition according to item 1, wherein the active ingredient or pharmaceutically acceptable salt thereof is felodipine or a pharmaceutically acceptable salt thereof.
3. The composition according to any one of items 1 to 2, wherein the HPMC-AS and/or hydroxypropyl methyl cellulose is present in a total amount of about 8% to 14% by weight.
4. The composition according to any one of the preceding items, wherein the PEO is present in an amount of about 85 to 95% by weight.
5. The composition according to any one of the preceding items, wherein the composition does not comprise hydroxypropyl methyl cellulose.
6. The composition according to any one of the preceding items, wherein the composition does not comprise a lubricant.
7. The composition according to any one of the preceding items, wherein the active ingredient is present in an amount equal to, or lower than 5% by weight.
8. The composition according to any one of the preceding items, wherein the composition is in a solid dosage form.
9. The composition according to item 8, wherein the felodipine is present in an amount between 0.05 mg and 10 mg of the solid dosage form.
10. The composition according to any one of the preceding items, wherein the composition is able to increase tₘₐₓ of the active ingredient, as compared to an equivalent amount of the active ingredient administered as an immediate release formulation.
11. The composition according to any one of the preceding items, wherein the composition is able to increase the dissolution rate of the active ingredient compared to a composition comprising a crystalline form of the active ingredient.
12. A method of manufacturing a composition comprising the steps of:
   a. preparing a melt uniform dispersion comprising felodipine or a pharmaceutically acceptable salt thereof together with HPMC and/or HPMC-AS, and
   b. cooling the melt to obtain a solid composition, wherein at least a portion of the active ingredient is in amorphous form.
13. The method according to item 12, further comprising a step of combining the composition of b. with PEO.
14. A composition according to any one of items 1 to 11, for use as a medicament.
15. A composition according to any one of items 1 to 11 or 14, for use in the treatment, prevention or alleviation of a hypertensive disease, angina pectoris, myocardial infarction or stroke.

### EXAMPLES

### Example 1: Preparation of Compositions and Study of Release Rate

### Aim

To illustrate the preparation of compositions according to the present disclosure and study the effect on drug release.

### Materials and Methods

Two different 3×8 mm, 200 mg mass tablet compact formulations of felodipine were produced and tested in triplicate using a standard dissolution USP2 apparatus. The tablets contained 5 mg felodipine and a ratio of 90:10 PEO:HPMC-AS and were prepared as described below.

Test sample: An amorphous solid dispersion is prepared by fusion in an electric furnace (simulating hot melt extrusion process) by heating a physical mixture of felodipine and HPMC-AS to around 160° C. followed by rapid cooling and grinding. This amorphous powder is then mixed with PEO and compacted.

Control sample: The control tablet is prepared by direct compression a crystalline physical mixture of felodipine and HPMC-AS compacted with PEO.

Dissolution of the tablets takes place in USP2 vessels with 250 mL 0.1 M phosphate buffer pH 6.8 at 50 rpm. Samples will be taken at defined time points and analyzed for felodipine content using HPLC-UV.

### Results

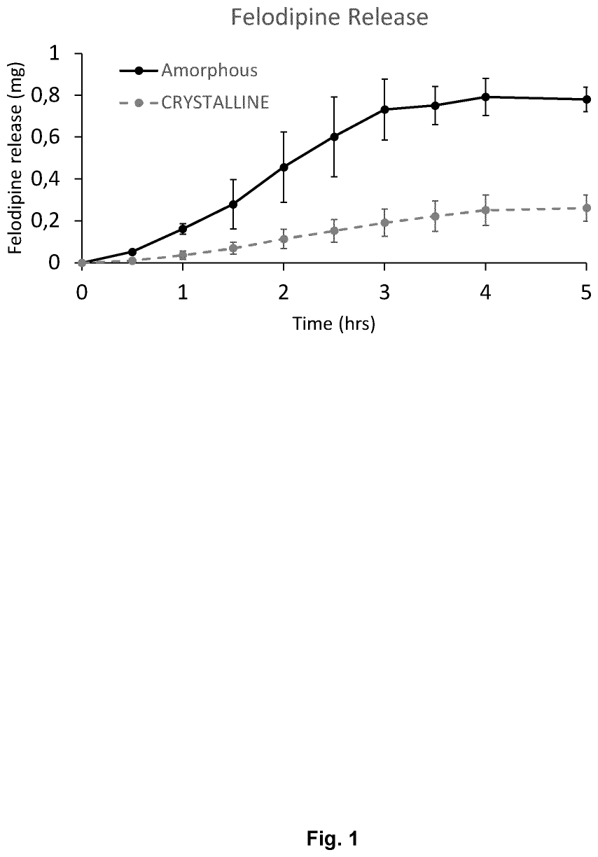

As shown in FIG. 1, it was observed that the compositions comprising amorphous felodipine display an enhanced dissolution rate.

### Conclusion

Formulations according to the present disclosure provide for sustained felodipine release and compositions comprising amorphous felodipine display an increased dissolution rate.

### Example 2: IVIVC Correlations

### Aim

To demonstrate the ability of the compositions according to the present disclosure to provide sustained release of felodipine.

### Materials and Methods

The in vitro in vivo correlation (IVIVC) method uses in vitro dissolution data to derive blood drug levels using the pharmacokinetic parameters of a test product. The convolution approach starts with dissolution results or profiles and develops the in vivo or drug concentration-time estimated profiles. Thus, using the dissolution rate data obtained in example 1 with felodipine, the expected profiles were modeled using a well-validated method as described in Qureshi et al. 20103.

Briefly, these profiles are converted into discrete dosage segments. The half-life (t½) of felodipine is estimated to be 9 h, Volume of distribution (Vd) is 10 L/kg for body weight (BW) of 75 kg. Under the assumption that felodipine is fastly absorbed through the intestine, the corresponding fold increase in dissolution rate observed in Example 1 was incorporated in the assumed bioavailabilities for crystalline and amorphous formulations, respectively.

### Results

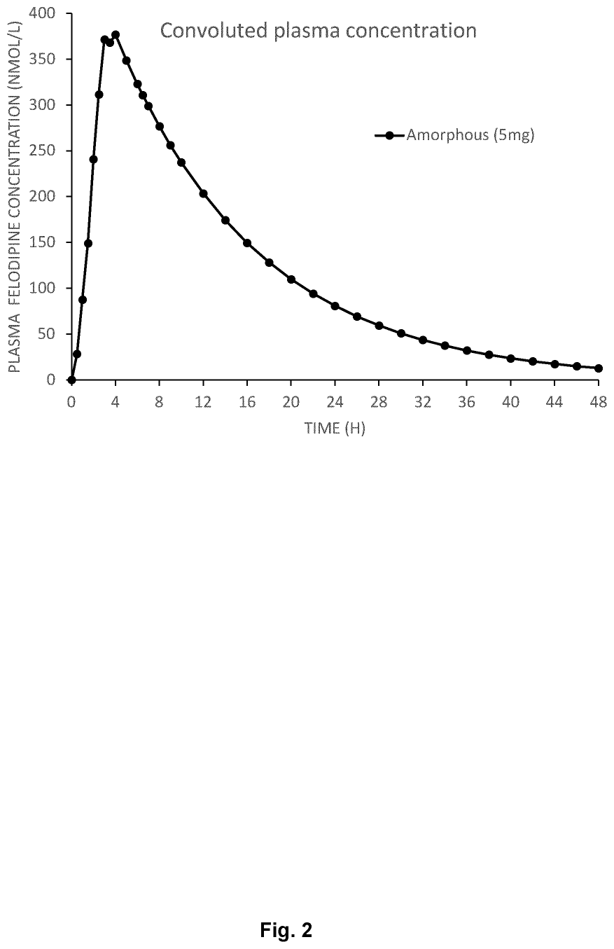

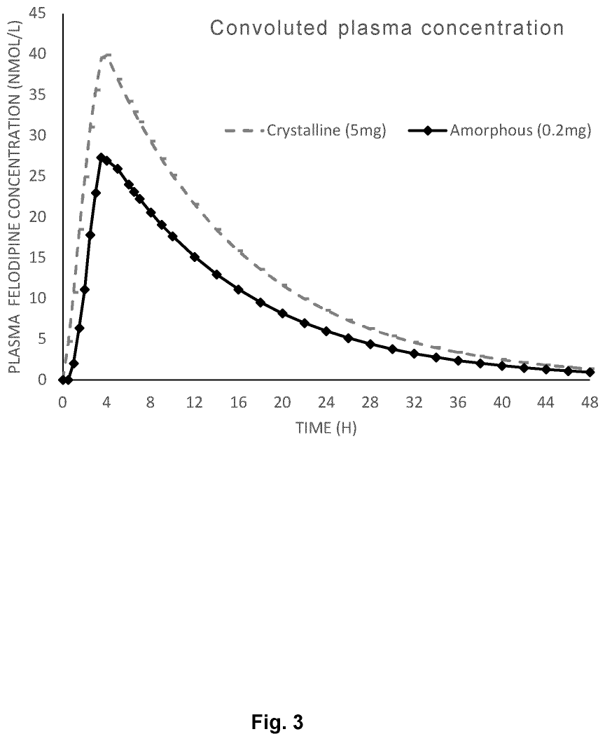

The convoluted predicted felodipine serum plasma profile for the amorphous composition 5 mg based on the test sample is displayed in FIG. 2. FIG. 3 shows a comparison between the predicted serum profiles of the 5 mg crystalline felodipine compositions (control sample) and the predicted profile for a hypothetical 0.2 mg dose of an amorphous sample. For both compositions, it is observed that Tₘₐₓ is around 4 hours.

### Conclusion

The enhanced dissolution rate of the amorphous felodipine composition translates into an expected increase in bioavailability. Thus, highlighting that the compositions according to the present can achieve equivalent bioavailability with lower doses, while providing with sustained release.

The dissolution rate provided achieves a drug release that achieve peak concentration of the active ingredients with a tₘₐₓ of between 3 to 6 hours with a safe return to baseline. This is specifically advantageous for administration of active ingredients that regulate events which increase or decrease during the night, for example blood pressure.

### REFERENCES

1. Douma L G, Gumz M L. Circadian clock-mediated regulation of blood pressure. Free Radic Biol Med. 2018 May 1; 119:108-114. doi: 10.1016/j.freeradbiomed.2017.11.024. Epub 2017 Dec. 2. PMID: 29198725; PMCID: PMC5910276.
2. M9 Biopharmaceutics Classification System-Based Biowaivers. Guidance for Industry. May 2021. FDA docket number: FDA-2018-D-3614.
3. Qureshi S A. In Vitro-In Vivo Correlation (IVIVC) and Determining Drug Concentrations in Blood from Dissolution Testing—A Simple and Practical Approach. The Open Drug Delivery Journal, 2010, Volume 4

1. A composition comprising:
i. hydroxypropyl methyl cellulose acetate succinate (HPMC-AS) and/or hydroxypropyl methyl cellulose in a total amount of 0.1% to 20% by weight, and
ii. polyethylene oxide (PEO) in an amount from 80 to 99.9% by weight, and
iii. an active ingredient or a pharmaceutically acceptable salt thereof;
wherein said active ingredient is a class II or class IV substance in the Biopharmaceutics Classification System (BCS), and wherein at least a portion of said active ingredient or pharmaceutically acceptable salt thereof is in an amorphous form.

2. The composition according to any one of the preceding claims, wherein the HPMC-AS is present in an amount of about 1% to 20%, such as about 2% to 20%, such as about 5% to 15%, such as about 6% to 14%, such as about 7% to 13%, such as about 8% to 12%, such as about 9% to 11%, for example about 10%.

3. The composition according to any one of the preceding claims, wherein the hydroxypropyl methyl cellulose is present in an amount of about 1% to 20%, such as about 2% to 20%, such as about 5% to 15%, such as about 6% to 14%, such as about 7% to 13%, such as about 8% to 12%, such as about 9% to 11%, for example about 10%.

4. The composition according to any one of the preceding claims, wherein the HPMC-AS and/or hydroxypropyl methyl cellulose is present in a total amount of about 1% to 20%, such as about 2% to 20%, such as about 5% to 15%, such as about 6% to 14%, such as about 7% to 13%, such as about 8% to 12%, such as about 9% to 11%, for example about 10%.

5. The composition according to any one of the preceding claims, wherein the PEO has an average molecular weight between 10 kDa to 2000 kDa, such as 50 kDa to 1000 kDa, such as 100 kDa to 500 kDa, such as 150 kDa to 300 kDa.

6. The composition according to any one of the preceding claims, wherein PEO is present in an amount of about 80 to 99%, such as about 85% to 95%, such as about 88% to 92%, such as about 90%.

7. The composition according to any one of the preceding claims, wherein the PEO is present in an amount of about 85 to 95%, such as 85% to 86%, such as 86% to 87%, such as 87% to 88%, such as 88% to 89%, such as 89% to 90%, such as 90% to 91%, such as 91% to 92%, such as 92% to 93%, such as 93% to 94%, such as 94% to 95%.

8. The composition according to any one of the preceding claims, wherein the components a), b) and c) are in a single matrix.

9. The composition according to any one of the preceding claims, wherein the weight ratio between PEO and the total amount of HPMC and/or HPMC-AS is between about 8:2 and about 9:0.1.

10. The composition according to any one of the preceding claims, wherein the composition comprises a uniform dispersion of the active ingredient with the HPMC and/or HPMC-AS; or with the PEO.

11. The composition according to any one of the preceding claims, wherein at least a portion of the active ingredient is in an amorphous solid dispersion.

12. The composition according to any one of the preceding claims, wherein the composition does not comprise HPMC or wherein the composition does not comprise HPMC-AS.

13. The composition according to any one of the preceding claims, wherein the composition is a solid dosage form.

14. The composition according to any one of the preceding claims, wherein the composition is compressed into a tablet.

15. The composition according to any of the preceding claims, further comprising one or more further polymers each independently selected from the group consisting of ionic, non-ionic, water-insoluble polymers, and water-soluble polymers.

16. The composition according to any one of the preceding claims, wherein the one or more further polymers are each independently selected from the group consisting of polysaccharides, polyacrylates and polysiloxanes and derivatives thereof.

17. The composition according to any one of the preceding claims, wherein the one or more further polymers are each independently selected from the group consisting of polyethylene oxide glucomannan, galactan, glucan, polygalacturonic acid, polyhydroxyalkanoates, polyxylane, polygalactomannans, rhanogalacturonan, polyxyloglycan, arabinogalactan, starch, alginates, xhanthan gum, carrageenan, agar, dextran, pectins, cellulose, polyvinyl alcohol, polyvinyl butyral, polyvinyl pyrrolidone, methylcellulose, ehtylcellulose, hydroxypropyl cellulose, hydroxypropyl methylcellulose stearate, carboxymethyl cellulose, carbomers, polyacrylic acid, poly(methylacrylic) acid, poly(methylmethacrylate), polyhydroxybutyrate, polyhydroxyvalerate, polyhydroxyphenylvalerate, polylactic acid, polyglycolic acid, a polyacrylic amide, and derivatives or copolymers thereof.

18. The composition according to any one of the preceding claims, wherein the composition does not comprise a lubricant.

19. The composition according to any one of the preceding claims, wherein the active ingredient has low water solubility.

20. The composition according to any one of the preceding claims, wherein the active ingredient is a class II substance in the Biopharmaceutics Classification System (BCS).

21. The composition according to any one of the preceding claims, wherein the active ingredient is a class IV substance in the Biopharmaceutics Classification System (BCS).

22. The composition according to any one of the preceding claims, wherein the active ingredient's highest dose is not soluble in 250 mL of water over the pH range of 1.0-7.5 at 37° C.

23. The composition according to any one of the preceding claims, wherein the active ingredient has a solubility in aqueous media, such as water or buffered saline below 0.1 g/L, such as below 0.05 g/L.

24. The composition according to any one of the preceding claims, wherein the active is an active ingredient having poor solubility according to the Biopharmaceutical classification systems (BCS) selected from any one of the group consisting of: afabicin, afabicin desphosphono, cobicistat, pralsetinib, piroxicam, tamoxifen glyburide, etodolac, tinidazole, bezafibrate, efavirenz, nevirapine, ergometrine, orismilast, nalmefene, asalhydromorphone, firategrast, lamotrigine, silibinin, hydrochlorothiazide, felodipine, posaconzaole, terbinafine, olorifim, cariprazine, tibolone, sertindole, daclatasvir, risperidone, pitavastatin, lovastatin, estradiol, raloxifene, mercaptopurine, atuliflapon, guanfacine, espirolactone, enzalutamide, firibastat, abrocitinib, glimpiride, ketoconazole, 1-methylnicotinamide, ibrutinib, rivaroxaban, safinamide, budesonide, ralinepag, valsartan, rimegepant, magnesium hydroxide, bicalutamide, vericiciguat, buphenine, bromocriptine, daridorexant, dipraglurant, dexlansoprazole, esomeprazole, sotagliflozin, duloxetine, belzutifan, sibutramine, loperamide, asciminib, basimglurant, deucravacitinib, fenofibrate, omeprazole, buprenorphine, rolapitant, lysine, finerenone, telmisartan, ansornitinib, mirtazapine, methimazole, temazepam, tadalafil, vardenafil, streptokinase, clopidogrel, nolatrexed, olanzapine, vilaprisan, cefuroxime, vapreotide, amprenavir, mefenamic acid, chlorzoxazone, drotaverine, butylscopolamine, danuglipron, palbociclib Isethionat, gepotidacin fostemsavir, calcium carbonate, pelubiprofen, zuclopentixol, zileuton, oxaprozin methylphenidate, phenazopyridine, flavoxate, methoxsalen, naproxen, lamotrigine, clenbuterol, hydroxyzine, loracarbef, ezogabine, phenytoin, racecadotril, crizotinib, griseofulvin, terbinafine, leniolisib, paliperidone, pemafibrate, nicardipine, amiodarone, carvedilol, methenamine, dicloxacillin, ketoconazole, tianeptine, onapristone, nitrofurantoin, loxapine, diacerein, bosentan, carbamazepine, omecamtiv mecarbil, oxacarbazepine, panobinostat, CE 224535 (CAS 724424-43-5), diclofenac, naltrexone, nicergoline, methocarbamol, metaxalone, chlorzoxazone, tizanidine, prednisolone, dipyridamole, mycophenolic acid, mycophenolate, felbamate, diosamin, sucralfate, gemfibrozil, carisoprodol, aplindore, cyclosporine, clonazepam, orlistat, sildenafil, lesinurad, cetylpyridinium, benzodiazepine, trimebutine, testosterone, betamethasone, gabapentin enacarbil, gabapentin, latanoprost, prochlorperazine, celecoxib, aceclofenac, nitazoxanide, dolutegravir, sulpiride, venetoclax, quinapril, cinacalcet, abiraterone, allopurinol, rifampicin, etravirine, acetazolamide, pyrantel, aripiprazole, sulfamethoxazole, venetoclax, ranolazine, folic acid, furosemide, azathioprine, chlorthalidone, adrulipase alfa, mifepristone, camlipixant, cethromycin, eritromycin, mesalazine, ritonavir, cisapride, apremilast, tannic acid, tolvaptan, canagliflozin, digoxin, cisplatin, ticagrelor, rifaximin, aprazolam, ketamine, albendazole, axitinib, metformin, chloropyramine, cinnarizine, clarithromycin, danazol, dextromethorphan, dimeticone, dronabinol, tramadol, dyclonine, aluminiumhydroxide, indomethacin, ketoprofen, ketorolac, letermovir, lornoxicam, maribavir, mebendazole, nicerogline, oxaprozin, oxcarbazepine, pancreatin, pemigatinib, pinaverium, pioglitazone, scopolamine, and selexipag, or a pharmaceutically acceptable salt thereof.

25. The composition according to any one of claims 1 to 24, wherein the active ingredient is a BCS Class II substance selected from any one of the group consisting of: afabicin, afabicin desphosphono, cobicistat, pralsetinib, piroxicam, tamoxifen glyburide, etodolac, tinidazole, bezafibrate, efavirenz, nevirapine, ergometrine, orismilast, nalmefene, asalhydromorphone, firategrast, lamotrigine, silibinin, hydrochlorothiazide, felodipine, posaconzaole, terbinafine, olorifim, cariprazine, tibolone, sertindole, daclatasvir, risperidone, pitavastatin, lovastatin, estradiol, raloxifene, mercaptopurine, atuliflapon, guanfacine, espirolactone, enzalutamide, firibastat, abrocitinib, glimpiride, ketoconazole, 1-methylnicotinamide, ibrutinib, rivaroxaban, safinamide, budesonide, ralinepag, valsartan, rimegepant, magnesium hydroxide, bicalutamide, vericiciguat, buphenine, bromocriptine, daridorexant, dipraglurant, dexlansoprazole, esomeprazole, sotagliflozin, duloxetine, belzutifan, sibutramine, loperamide, asciminib, basimglurant, deucravacitinib, fenofibrate, omeprazole, buprenorphine, rolapitant, lysine, finerenone, telmisartan, ansornitinib, mirtazapine, methimazole, temazepam, tadalafil, vardenafil, streptokinase, clopidogrel, nolatrexed, olanzapine, vilaprisan, cefuroxime, vapreotide, amprenavir, mefenamic acid, chlorzoxazone, drotaverine, butylscopolamine, danuglipron, palbociclib lsethionat, gepotidacin fostemsavir, calcium carbonate, pelubiprofen, zuclopentixol, zileuton, oxaprozin methylphenidate, phenazopyridine, flavoxate, methoxsalen, naproxen, lamotrigine, clenbuterol, hydroxyzine, loracarbef, ezogabine, phenytoin, racecadotril, crizotinib, griseofulvin, terbinafine, leniolisib, paliperidone, pemafibrate, nicardipine, amiodarone, carvedilol, methenamine, dicloxacillin, ketoconazole, tianeptine, onapristone, nitrofurantoin, loxapine, diacerein, bosentan, carbamazepine, omecamtiv mecarbil, oxacarbazepine, panobinostat, CE 224535 (CAS 724424-43-5), diclofenac, naltrexone, nicergoline, methocarbamol, metaxalone, chlorzoxazone, tizanidine, prednisolone, dipyridamole, mycophenolic acid, mycophenolate, felbamate, diosamin, sucralfate, gemfibrozil, carisoprodol, aplindore, cyclosporine, clonazepam, orlistat, sildenafil, lesinurad, cetylpyridinium, benzodiazepine, trimebutine, testosterone, betamethasone, gabapentin enacarbil, gabapentin, latanoprost, prochlorperazine, celecoxib, aceclofenac, nitazoxanide, and dolutegravir, albendazole, axitinib, metformin, chloropyramine, cinnarizine, clarithromycin, danazol, dextromethorphan, dimeticone, dronabinol, tramadol, dyclonine, aluminiumhydroxide, indomethacin, ketoprofen, ketorolac, letermovir, lornoxicam, maribavir, mebendazole, nicerogline, oxaprozin, oxcarbazepine, pancreatin, pemigatinib, pinaverium, pioglitazone, scopolamine, and selexipag, or a pharmaceutically acceptable salt thereof.

26. The composition according to any one of claims 1 to 24, wherein the active ingredient is a BCS Class IV substance selected from any one of the group consisting of: sulpiride, venetoclax, quinapril, cinacalcet, abiraterone, allopurinol, rifampicin, etravirine, acetazolamide, pyrantel, aripiprazole, sulfamethoxazole, venetoclax, ranolazine, folic acid, furosemide, azathioprine, chlorthalidone, adrulipase alfa, mifepristone, camlipixant, cethromycin, eritromycin, mesalazine, ritonavir, cisapride, apremilast, tannic acid, tolvaptan, canagliflozin, digoxin, cisplatin, ticagrelor, rifaximin, aprazolam, and ketamine, or a pharmaceutically acceptable salt thereof.

27. The composition according to any one of claims 1 to 24, wherein the active ingredient is is selected from any one of the group consisting of: afabicin, afabicin desphosphono, cobicistat, pralsetinib, piroxicam, tamoxifen glyburide, etodolac, tinidazole, bezafibrate, efavirenz, nevirapine, ergometrine, orismilast, nalmefene, asalhydromorphone, firategrast, lamotrigine, silibinin, hydrochlorothiazide, felodipine, posaconzaole, terbinafine, olorifim, cariprazine, tibolone, sertindole, daclatasvir, risperidone, pitavastatin, lovastatin, estradiol, raloxifene, mercaptopurine, atuliflapon, guanfacine, espirolactone, enzalutamide, firibastat, abrocitinib, glimpiride, ketoconazole, 1-methylnicotinamide, ibrutinib, rivaroxaban, safinamide, budesonide, ralinepag, valsartan, rimegepant, magnesium hydroxide, bicalutamide, vericiciguat, buphenine, bromocriptine, daridorexant, dipraglurant, dexlansoprazole, esomeprazole, sotagliflozin, duloxetine, belzutifan, sibutramine, loperamide, asciminib, basimglurant, deucravacitinib, fenofibrate, omeprazole, buprenorphine, rolapitant, lysine, finerenone, telmisartan, ansornitinib, mirtazapine, methimazole, temazepam, tadalafil, vardenafil, streptokinase, clopidogrel, nolatrexed, olanzapine, and vilaprisan, or a pharmaceutically acceptable salt thereof.

28. The composition according to any one of claims 1 to 24, wherein the active ingredient is selected from any one of the group consisting of: cefuroxime, vapreotide, amprenavir, mefenamic acid, chlorzoxazone, drotaverine, butylscopolamine, danuglipron, palbociclib lsethionat, gepotidacin fostemsavir, calcium carbonate, pelubiprofen, zuclopentixol, zileuton, oxaprozin methylphenidate, phenazopyridine, flavoxate, methoxsalen, naproxen, lamotrigine, clenbuterol, hydroxyzine, loracarbef, ezogabine, phenytoin, racecadotril, crizotinib, griseofulvin, terbinafine, leniolisib, paliperidone, pemafibrate, nicardipine, amiodarone, carvedilol, methenamine, dicloxacillin, ketoconazole, tianeptine, onapristone, nitrofurantoin, loxapine, diacerein, bosentan, carbamazepine, omecamtiv mecarbil, oxacarbazepine, panobinostat, CE-224535 (CAS 724424-43-5), diclofenac, naltrexone, nicergoline, methocarbamol, metaxalone, chlorzoxazone, tizanidine, prednisolone, dipyridamole, mycophenolic acid, mycophenolate, felbamate, diosamin, sucralfate, gemfibrozil, carisoprodol, aplindore, cyclosporine, clonazepam, orlistat, sildenafil, lesinurad, cetylpyridinium, benzodiazepine, trimebutine, testosterone, betamethasone, gabapentin enacarbil, gabapentin, latanoprost, prochlorperazine, celecoxib, aceclofenac, nitazoxanide, and dolutegravir, or a pharmaceutically acceptable salt thereof.

29. The composition according to any one of claims 1 to 24, wherein the active ingredient is selected from any one of the group consisting of: sulpiride, venetoclax, quinapril, cinacalcet, abiraterone, allopurinol, rifampicin, etravirine, acetazolamide, pyrantel, aripiprazole, sulfamethoxazole, venetoclax, ranolazine, folic acid, furosemide, azathioprine, chlorthalidone, adrulipase alfa, and mifepristone, or a pharmaceutically acceptable salt thereof.

30. The composition according to any one of claims 1 to 24, wherein the active ingredient is selected from any one of the group consisting of: camlipixant, cethromycin, eritromycin, mesalazine, ritonavir, cisapride, apremilast, tannic acid, tolvaptan, canagliflozin, digoxin, cisplatin, ticagrelor, rifaximin, aprazolam, and ketamine, or a pharmaceutically acceptable salt thereof.

31. The composition according to any one of the preceding claims, wherein the active ingredient is present in an amount lower equal or lower than 10% by weight, such as lower than 9%, 8%, 7%, 6% or 5% by weight.

32. The composition according to any one of the preceding claims, wherein the active ingredient is present in an amount between 0.001 mg and 200 mg.

33. The composition according to any one of the preceding claims, wherein the active ingredient that regulates cardiac events or cardiovascular events.

34. The composition according to any one of the preceding claims, wherein the the active ingredient is an active ingredient that regulates cardiac events or cardiovascular events, that benefits from chronotherapy.

35. The composition according to any one of the preceding claims, wherein the active ingredient is selected form the group consisting of: felodipine, nicardipine, valsartan, telmisartan, chlorathalidone, digoxin, and temazepam, or a pharmaceutically acceptable salt thereof.

36. The composition according to any one of claims 1 to 34, wherein the active ingredient is an antihypertensive.

37. The composition according to claim 36, wherein the antihypertensive is selected form the group consisting of: felodipine, nicardipine, valsartan, and telmisartan, or a pharmaceutically acceptable salt thereof.

38. The composition according to any one of the preceding claims, wherein the active ingredient is selected from the group consisting of: felodipine, itraconazole and naproxen.

39. The composition according to any one of the preceding claims, wherein the active ingredient is felodipine or a pharmaceutically acceptable salt thereof.

40. The composition according to any one of claims 1 to 32, wherein the active ingredient is naproxen or a pharmaceutically acceptable salt thereof.

41. The composition according to any one of claims 1 to 32, wherein the active ingredient is itraconazole or a pharmaceutically acceptable salt thereof.

42. The composition according to any one of claims 1 to 32, wherein the active ingredient is dotraverine, or a pharmaceutically acceptable salt thereof, such as dotraverine hydrochloride.

43. The composition according to any one of claims 1 to 32, wherein the active ingredient is butylscopolamine, or a pharmaceutically acceptable salt thereof, such as butylscopolamine bromide.

44. The composition according to any one of claims 1 to 32, wherein the active ingredient is calcium carbonate.

45. The composition according to any one of claims 1 to 32, wherein the active ingredient is cethromycin, or a pharmaceutically acceptable salt thereof.

46. The composition according to any one of claims 1 to 32, wherein the active ingredient is pelubiprofen, or a pharmaceutically acceptable salt thereof.

47. The composition according to any one of claims 1 to 32, wherein the active ingredient is flavoxate, or a pharmaceutically acceptable salt thereof.

48. The composition according to any one of claims 1 to 32, wherein the active ingredient is phenazopyridine, or a pharmaceutically acceptable salt thereof.

49. The composition according to claim 39, wherein the felodipine is present in an amount between 0.05 mg and 10 mg.

50. The composition according to claim 40, wherein the naproxen is present in an amount between 1 to 1500 mg.

51. The composition according to claim 41, wherein the itraconazole is present in an amount between 1 mg and 300 mg.

52. The composition according to any one of the preceding claims, wherein the composition is able to increase the dissolution rate of the active ingredient.

53. The composition according to any one of the preceding claims, wherein the composition is able to increase the dissolution rate of the active ingredient compared to a composition comprising a crystalline form of the active ingredient.

54. The composition according to any one of the preceding claims, wherein the composition is able to increase the absorption rate of the active ingredient in the gut.

55. The composition according to any one of the preceding claims, wherein the composition is able to increase the oral bioavailability of the active ingredient compared to a composition comprising a crystalline form of the active ingredient.

56. The composition according to any one of the preceding claims, wherein the composition is able to increase tₘₐₓ of the active ingredient, as compared to an equivalent amount of the active ingredient administered as an immediate release formulation.

57. The composition according to any one of the preceding claims, wherein tₘₐₓ of the active ingredient is increased by at least about 1 hour, such as at least about 2 hours, for example at least about 3 hours, such as at least about 4 hours as compared to an equivalent amount of the active ingredient administered as an immediate release formulation.

58. The composition according to any one of the preceding claims, wherein the composition provides tₘₐₓ of the active ingredient of 3 hours to 6 hours upon oral administration, such as tₘₐₓ of 3.0, 3.5, 4.0, 4.5, 5.0, 5.5, or 6.0 hours.

59. The composition according to any one of the preceding claims, wherein the composition provides tmax of the active ingredient of from 3.0 to 4.0 hours, or of from 4.0 to 5.0 hours, or of from 5.0 to 6.0 hours.

60. The composition according to any one of the preceding claims, wherein the composition is to be administered between 18 h and 00 h, such as between 20 h and 00 h, for example at 20 h, 21 h, 22 h, 23 h or 00 h.

61. The composition according to any one of the preceding claims, wherein the composition is to be administered during dinner or after dinner, such as within 3 hours after dinner, such as within 2 hours after dinner, such as within 1 hour after dinner, such as within 30 minutes after dinner.

62. The composition according to any one of the preceding claims, wherein the composition is to be administered prior to a major sleep episode, such as within 3 hours before a major sleep episode, such as within 2 hours before a major sleep episode, for example within 1 hour before a major sleep episode, such as within 30 minutes before a major sleep episode.

63. The composition according to any one of the preceding claims, wherein the composition is for oral administration.

64. The composition according to any one of the preceding claims, wherein the composition is a single-unit oral dosage form, such as a pill, a tablet, a thermoformed unit or a capsule.

65. The composition according to any one of the preceding claims, wherein the composition is to be administered on an empty stomach.

66. The composition according to any one of the preceding claims, wherein the composition is to be administered once daily.

67. The composition according to any one of claims 1 to 65, wherein the composition is to be administered more than once daily, such as 2 times daily, 3 times daily, or 4 times daily.

68. A composition comprising a matrix comprising:
i. an active ingredient or a pharmaceutically acceptable salt thereof as described herein,
ii. HPMC and/or HPMC-AS; and
iii. polyethylene oxide (PEO),
wherein the weight ratio of PEO (component iii.) to HPMC and/or HPMC-AS (component ii.) is between 8:2 and 9:0.1, and
wherein at least a portion of the active ingredient or pharmaceutically acceptable salt thereof is in an amorphous form.

69. The composition according to claim 68, wherein the active ingredient is according to any one of claims 19 to 51.

70. The composition according to any one of claims 68 to 69, wherein the PEO, the HPMC and/or the HPMC-AS is according to any one of claims 2 to 12.

71. The composition according to any one of claims 68 to 70, wherein the composition comprises one or more further polymer(s) according to any one of claims 15 to 17.

72. The composition according to any one of claims 68 to 71, wherein the composition is a solid oral dosage form, such as a tablet.

73. A method of manufacturing a composition comprising the steps of:
a. preparing a melt uniform dispersion comprising an active ingredient or a pharmaceutically acceptable salt thereof together with HPMC or HPMC-AS, and
b. cooling the melt to obtain a solid composition, wherein at least a portion of the active ingredient is in amorphous form
c. combining the composition in b. with PEO;
wherein the final weight ratio between PEO and the HPMC and/or HPMC-AS in the composition is between 8:2 and 9:0.1.

74. The method according to claim 73, wherein step a. involves heating the mixture to a temperature wherein the active ingredient becomes soluble in the HPMC and/or HPMC-AS.

75. The method according to any one of claims claims 73 to 74, wherein step a. further comprises PEO.

76. The method according to any one of claims claims 73 to 75, wherein step a. involves heating the mixture to a temperature wherein the active ingredient becomes soluble in the mixture of HPMC and/or HPMC-AS with PEO.

77. The method according to any one of claims 73 to 76, wherein step a. involves heating the mixture to a temperature wherein the active ingredient becomes amorphous.

78. The method according to any one of claims 73 to 77, wherein the melt in step a. is prepared by heating to a temperature above the melting temperature (Tₘ) of one of the matrix components.

79. The method according to any one of claims 73 to 78, wherein the melt in step a. is prepared by heating the active ingredient and HPMC-AS above 138° C., such as from 140° C. to 190° C., for example from 140° C. to 180° C., for example from 140° C. to 170° C., such as 140° C., 150° C., or 160° C.

80. The method according to any one of claims 73 to 79, further comprising a step of compressing the composition into a tablet.

81. The method according to any one of claims 73 to 80, wherein the active ingredient, the HPMC, the HPMC-AS and the PEO are as defined in any one of claims 1 to 57.

82. A composition according to any one of the claims 1 to 67, for use as a medicament.

83. Use of a composition according to any one of claims 1 to 67 in the manufacture of a medicament.

84. A method of treatment of a disease, said method comprising administering a composition according to any one of claims 1 to 67 to a subject in need thereof.

85. The composition according to any one of claims 1 to 37, 39, 49 and 52 to 67 for use in the treatment, prevention, or alleviation of an hypertensive disease, angina pectoris, myocardial infarction, or stroke.

86. The composition for use according to claim 85, wherein the hypertensive disease is selected from essential hypertension, hypertensive heart disease, hypertensive crisis or secondary hypertension.

87. The composition for use according to claim 85, wherein the myocardial infarction is selected from myocardial infarction is acute myocardial infarction or subsequent myocardial infarction.

88. The composition for use according to claim 85, wherein the angina pectoris is unstable or stable angina, angina syndrome, or ischaemic chest pain.

89. Use of the composition according to any one of claims 1 to 37, 39, 49 and 52 to 67 in the manufacture of a medicament for the treatment, prevention, or alleviation of a hypertensive disease, angina pectoris, or acute myocardial infarction.

90. A method of treatment, prevention or alleviation of an hypertensive disease, angina pectoris, or acute myocardial infarction, said method comprising administering the composition according to any one of claims 1 to 37, 39, 49 and 52 to 67 to a subject in need thereof.

91. The composition according to any one of claims 1 to 32, 40, 50, and 52 to 67 for use in the treatment, prevention or alleviation of pain such as pain associated with common cold, headache, toothache, muscular aches, or backache; inflammatory diseases, such as rheumatoid arthritis, osteoarthritis, ankylosing spondylitis, juvenile arthritis, tendonitis, bursitis or acute gout; dysmenorrhea or fever.

92. The composition according to any one of claims 1 to 32, 41, 51 and 52 to 67 for use in the treatment, prevention, or alleviation of a fungal infection, such as blastomycosis, histoplasmosis, aspergillosis, or onychomycosis.
